(19) United States
(12) Patent Application Publication
(10) Pub. No.: US 2025/0058113 A1
(43) Pub. Date: Feb. 20, 2025

(54) LINKING PULSE PATTERNS IN A NEUROSTIMULATION DEVICE

(71) Applicant: Boston Scientific Neuromodulation Corporation, Valencia, CA (US)

(72) Inventor: Philip Leonard Weiss, Sherman Oaks, CA (US)

(73) Assignee: Boston Scientific Neuromodulation Corporation, Valencia, CA (US)

(21) Appl. No.: 18/802,826

(22) Filed: Aug. 13, 2024

Related U.S. Application Data
(60) Provisional application No. 63/532,625, filed on Aug. 14, 2023.

(51) Int. Cl. A61N 1/36; A61N 1/05

(52) U.S. Cl. CPC A61N 1/36062; A61N 1/0551; A61N 1/36146

(57) ABSTRACT

An implantable neurostimulation device is configured with a current delivery architecture having at least two digital to analog converters (DACs), and a therapy definition circuit using therapy configurations is provided that allows two therapy configurations to be linked together. When two therapy configurations are linked, each therapy configuration uses a different one of the DACs to deliver therapy in overlapping or simultaneous fashion, using identical or different electrodes.

### CROSS-REFERENCE TO RELATED APPLICATIONS

This application claims the benefit of U.S. Provisional Patent Application Ser. No. 63/532,625, filed Aug. 14, 2023, which is incorporated herein by reference.

### BACKGROUND

Arbitration logic and/or instructions are used in implantable medical devices to determine the order of execution of multiple therapy programs. For example, US PG Pub. No. 2013/0184794 describes output circuitry for use in neuromodulation devices, such as spinal cord stimulation (SCS) or deep brain stimulation (DBS) devices. The devices may be programmed to issue multiple therapy programs to different or overlapping sets of therapy electrodes. With multiple therapy programs scheduled to issue therapy outputs using the overlapping electrodes and/or output circuitry, timing controls are used so that contrary instructions do not reach the same output defining circuits at the same time. For example, a single output digital to analog converter (DAC) could be commanded to issue a positive one milliamp current at the same time as it is commanded to issue a negative two milliamp current, causing problems with both of the competing therapy programs.

As more sophisticated therapy programs are desired with current and future systems, new and/or alternative arbitration schemes are desired. Of interest are approaches in which multiple pulse patterns can be linked together for therapy delivery purposes, without sacrificing the ability to arbitrate other pulse patterns.

### OVERVIEW

The present inventors have recognized, among other things, that a problem to be solved is the need for new and/or alternative arbitration schemes are desired. In illustrative examples, a system for therapy definition is used in which therapy phases are defined at a granular level by pulse instructions, grouped together as aggregate instructions which pair pulse instructions with electrode steering instructions, and the aggregate instructions are further grouped together by therapy configurations. Methods and devices allowing different sets of pulses, such as different combinations of aggregate instructions or therapy configurations together are disclosed.

A first illustrative and non-limiting example takes the form of an implantable medical device comprising: a housing containing a power source, a controller, and stimulation circuitry, the stimulation circuitry including at least first and second digital-to-analog converters (DACs); and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output current, and the steering logic is configured to implement a selected steering instruction set; wherein the pulse memory contains pulse programs, each having a one or more pulse instructions defining pulse components each having a pulse type and one or more determining characteristics for the pulse type; wherein the aggregate memory contains aggregate instructions each defining one or more aggregated outputs, each aggregated output pairing a selected steering instruction set with a selected pulse program and defining a number of repetitions for the selected pulse program to execute with the selected steering instruction set; wherein the configuration memory defines a plurality of therapy configurations each having a defined total stimulus output current, an arbitration mode, a holdoff setting, and linking data, and identifying a one or more aggregate instructions to be executed for each therapy configuration; further wherein the linking data in the configuration memory enables two therapy configurations to operate simultaneously, using separate DACs and any of the electrodes.

Additionally or alternatively, the linking data of a first therapy configuration identifies a second therapy configuration with which the first therapy configuration is linked.

Another illustrative, non-limiting example takes the form of aa implantable medical device comprising: a housing containing a power source, a controller, and stimulation circuitry, the stimulation circuitry including at least first and second digital-to-analog converters (DACs); and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; and a plurality of pulse definition circuits each including steering logic, aggregate logic, and pulse logic; wherein the steering memory contains steering instruction sets for a plurality of steering programs, each steering program determining which of the electrodes receive a fraction of a total stimulus output current, and the steering logic is configured to implement a selected steering instruction set; wherein the pulse memory contains pulse programs, each having a one or more pulse instructions defining pulse components each having a pulse type and one or more determining characteristics for the pulse type; wherein the aggregate memory contains aggregate instructions each defining one or more aggregated outputs, each aggregated output pairing a selected steering instruction set with a selected pulse program and defining a number of repetitions for the selected pulse program to execute with the selected steering instruction set; wherein the configuration memory defines a plurality of therapy configurations each having a defined total stimulus output current, an arbitration mode, a holdoff setting, and identifying a one or more aggregate instructions to be executed for each therapy configuration; the implantable medical device further comprising a scheduler identifying therapy configurations to be executed and times at which the therapy configurations are to be executed, wherein the scheduler is configured to identify a first therapy configuration as linked to a second therapy configuration, and to instruct the pulse definition circuits to simultaneously execute the first therapy configuration on a first DAC, and the second therapy configuration on a second DAC.

Additionally or alternatively, the first therapy configuration calls for issuance of a first therapy pulse from the first DAC having a first amplitude via a first electrode, and the second therapy configuration calls for issuance of a second therapy pulse simultaneous with the first therapy pulse, from the second DAC having a second amplitude via the first electrode. Additionally or alternatively, the first therapy configuration requests a first total current from the first DAC, and the second therapy configuration requests a second total current from the second DAC, wherein the first total current is larger than the second total current. Additionally or alternatively, the first therapy configuration calls for a first pulse train having first parameters, and the second therapy configuration calls for a second pulse train having second parameters, the first parameters being different from the second parameters. Additionally or alternatively, the stimulation circuitry comprises a plurality of digital-to-analog converter circuits including selectable current mirrors, and the total output amplitude is defined in terms of total output current, such that the implantable medical device is configured to deliver current controlled neural stimulation.

Additionally or alternatively, the pulse definition circuit is coupled to the plurality of digital-to-analog converter circuits and is configured to instruct a selected one of the plurality of digital-to-analog converter circuits to use the total output current, and divide the total output current using the selected steering instruction set. Additionally or alternatively, the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

Additionally or alternatively, the first therapy configuration and the second therapy configuration each reference a same steering program. Additionally or alternatively, the first therapy configuration and the second therapy configuration are determined by applying a cost function to approximate a desired therapy output using the first therapy configuration and the second therapy configuration. Additionally or alternatively, the first therapy configuration uses a different steering program than the second steering program. Additionally or alternatively, the first therapy configuration and the second therapy configuration are each current controller therapy configurations.

Some implementations may be in the form of an implantable deep brain stimulation system comprising the implantable medical device of any of the preceding examples, and a clinician programmer adapted to communicate with the implantable medical device and program each of the arbitration mode and holdoff settings stored in the implantable medical device; wherein the lead is adapted for placement in the brain of a patient.

Some implementations may be in the form of an implantable spinal cord stimulation system comprising the implantable medical device of any of the preceding examples, and a clinician programmer adapted to communicate with the implantable medical device and program each of the arbitration mode and holdoff settings stored in the implantable medical device; wherein the lead is adapted for placement in the spinal column of a patient.

Some implementations may be in the form of methods of operation of a device as in any of the preceding examples, including executing, for example, at least first and second therapy configurations at the same time.

Another illustrative and non-limiting example takes the form of a method of operation in an implantable neurostimulation device, the method comprising: calling for execution of a first therapy configuration according to a schedule, the first therapy configuration identifying a second therapy configuration as being linked thereto; executing the first therapy configuration on a first digital-to-analog converter (DAC) of the implantable neurostimulation device; and simultaneous to executing the first therapy configuration on the first DAC, executing a second therapy configuration on a second DAC; wherein: the first DAC, operating in accordance with the first therapy configuration, issues a first pulse using a first electrode of the implantable neurostimulation device during a first time period; and the second DAC, operating in accordance with the second therapy configuration, issues a second pulse using the first electrode during the first time period, such that the first and second pulses combine together as a single output pulse.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative stimulation system;

FIG. 2 shows additional details of a pulse generator;

FIG. 3 shows an illustrative prior art arbitration scheme;

FIG. 4 illustrate components or functional blocks of a stimulation control circuit;

FIG. 5 illustrates a steering program;

FIG. 6 illustrates a plurality of pulse phase types and an illustrative pulse program;

FIG. 7 shows illustrative aggregate instructions;

FIG. 8 shows illustrative therapy configurations with arbitration and linking; and

FIGS. 9-10 show illustrative linked therapy configurations.

### DETAILED DESCRIPTION

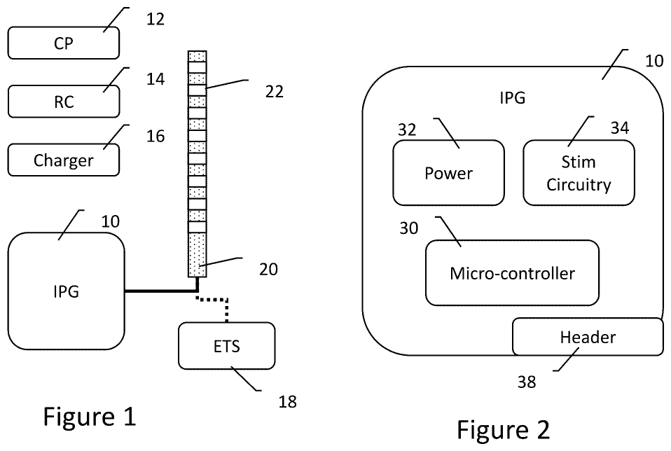

FIG. 1 shows an illustrative stimulation system. An implantable pulse generator (IPG) 10 is operably linked to a lead 20 having electrodes 22. The lead 20 may be placed in any suitable position, such as locations for use in DBS, SCS and/or other therapy including treating the vagus nerve, digestive/excretory tracts, etc., to bring the electrodes 22 into proximity to target tissue such as neural targets.

For DBS, the IPG 10 may be positioned, for example, in the upper chest of a patient, with the lead 20, possibly including a lead extension, extending beneath the skin to the head of the patient, where a bore hole through the skull is prepared and the lead is then passed into the brain near a target structure, such as the thalamus, subthalamic nucleus, globus pallidus, or other structures. A lead used in DBS may include a combination of segmented and ring electrodes 22, if desired, such as disclosed in U.S. Pat. Nos. 8,483,237 and 8,321,025, the disclosures of which are incorporated herein by reference.

For SCS, the IPG may be positioned, without limitation, in the region of the buttocks, with the lead extending toward the thoracic spine, for example, such that one or more leads 20 are positioned therein; while a cylindrical lead 20 with a series of ring electrodes 22 is shown in FIG. 1, a paddle lead may be used instead for SCS, having columns of electrodes positioned side-by-side. In SCS, the lead will be extended into the vicinity of the spinal column, such as disclosed variously in any of U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference.

The IPG 10 can include communication circuitry using, for example and without limitation, Bluetooth, Medradio, or other communications modes, frequencies and standards, to communicate wirelessly while implanted to one or more of a clinician programmer (CP) 12, and a patient remote control (RC) 14. The CP 12 can be used by or at the direction of a physician to select various therapy parameters as is known in the art. The CP 12 may be used to set the various arbitration parameters and linking controls discussed below. The RC 14 can be used by the patient, typically, to turn therapy on and/or off, to interrogate the IPG 10 to determine device status, and sometimes to adjust therapy settings, such as by changing amplitude of stimulation, or provide patient feedback such as by answering a patient questionnaire.

A charger is shown at 16, and may be used to provide power to the IPG 10 for recharging the batteries of the IPG. Power may be, for example and without limitation, transmitted by inductive coupling between the charger 16 and IPG 10. The present invention is not limited to rechargeable IPGs 10, and may be used as well with non-rechargeable or “primary cell” IPGs 10, in which case the charger 16 may be omitted. An external test system (ETS) 18 is included. The ETS 18 can be programmed similar to the IPG 10, using, for example, the CP 12, and controlled with RC 14, if desired. The ETS 18 may be used to test therapy programs for efficacy on the patient after the lead 20 has been implanted and before implantation of the permanent IPG 10, as is well known in the art.

The plurality of electrodes 22 may be used to deliver targeted therapy in various ways. For example, with a current-controlled therapy, a total quantity of current to be issued via the electrodes can be fractionalized or divided amongst the electrodes to create a volume of activation for the therapy and/or define a central point of stimulation, using known methods. This may be referred to as current steering. For current steering, a system may have a plurality of independent current-controlled outputs, sometimes referred to as multiple independent current control. Voltage-controlled therapy may be issued instead, using a plurality of independent voltage outputs, also allowing therapy to be issued with some degree of control over where the therapy is targeted by selective use of the electrodes. While much of the following is discussed in the context of current-controlled stimulation, arbitration concepts as discussed herein can apply to either current-controlled or voltage-controlled therapy.

FIG. 2 shows additional details of a pulse generator. The IPG 10 may include separate circuits, sometimes referred to as operational circuitry, including a microcontroller 30 (which may also be implemented as part of a microprocessor if desired), which controls operations of the IPG at a high level. The IPG can include a power source 32, typically a battery (rechargeable or primary cell, as desired), though some systems may be adapted to operate without a battery by receiving power inductively or through other link (such as radiofrequency) and issuing therapy using the received power without long-term storage. The microcontroller may include memory for storing operational instructions in a non-transient media, such as a Flash memory, RAM, ROM, etc.

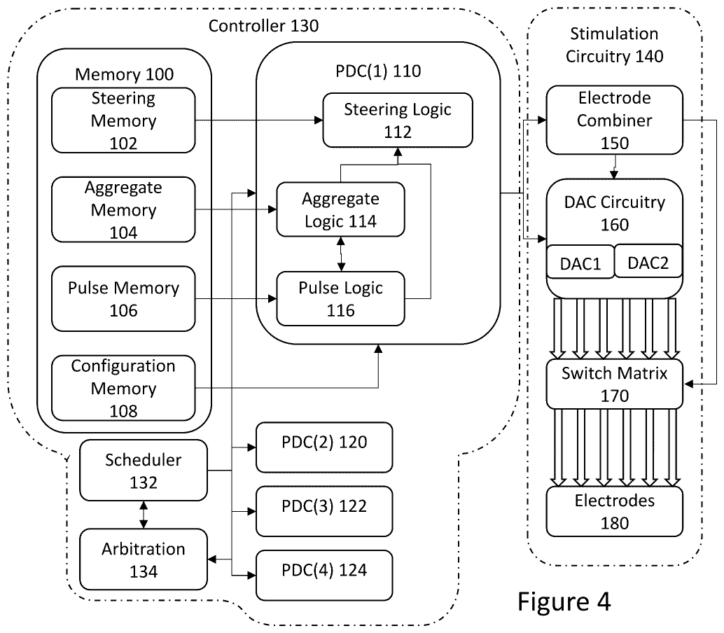

A block of stimulation circuitry 34 is also provided, and may include componentry and/or functional blocks as discussed below in relation to FIG. 4. At a high level the stimulation circuitry 34 may include a plurality of current sources and current sinks (for a current-controlled system; a plurality of voltage sources may be used in voltage-controlled systems instead), and control circuitry including for example one or more analog ASICs, as well as switch arrays that implement steering instructions and/or electrode selections. U.S. Pat. No. 10,716,932 provides illustrative details for both current and planned future implementations of the stimulation circuitry 34, and is incorporated herein by reference.

The IPG 10 may include a conductive outer housing that can serve as a return electrode or indifferent electrode during therapy delivery, as desired. A header 38 provides feedthrough circuitry allowing the IPG 10 to couple to a lead 20 (FIG. 1), with separate electrical connections to each of the electrodes 22. The lead 20 (FIG. 1) may further include any other desired components (sensors, and optical devices, for example), and the header 38 will include electrical connections for such other components as well. The header 38 and housing provide a hermetic sealed environment for the operational circuitry 30, 32, 34.

In some examples, a plurality of programs can be set for therapy delivery by the IPG 10. Each program may operate according to a schedule and individual program parameters. When a schedule causes two programs to request overlapping stimulus output, the system may use arbitration to determine which program will output stimulus first.

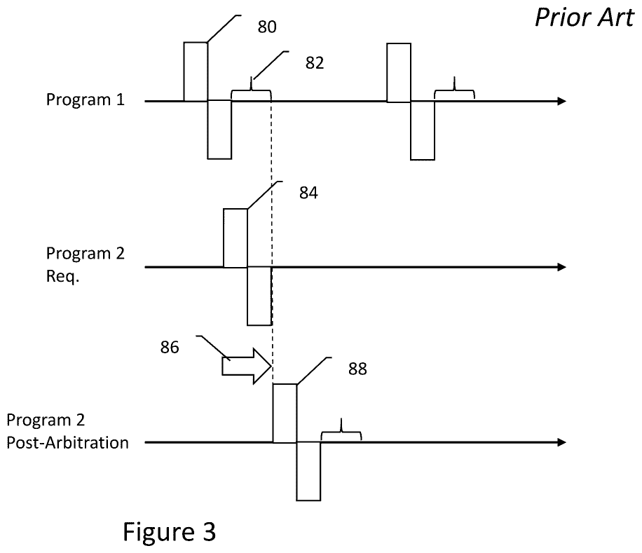

FIG. 3 shows an illustrative prior art arbitration scheme. Here, Program 1 provides a biphasic square wave output shown at 80, and includes a post-stimulation quiet (or quiescent) period at 82. The prior art arbitration logic enforces a hold-off until completion of the quiet period at 82. In the example, shown, Program 2 requests to use the output circuit to issue a therapy pulse as shown at 84. However, this request is denied because Program 1 is still using the output circuitry. The arbitration logic enforces a holdoff or delay, as shown at 86, and does not allow Program 2 to issue the therapy pulse until a later time, as shown at 88, following expiration of the hold-off for the quiet period 82. This arbitration logic prevents Program 2 from operating as intended, but defends the output circuitry from competing commands. With newer circuitry and more sophisticated therapy patterns/programs being studied, a more flexible system is desired.

FIG. 4 illustrates components or functional blocks of a stimulation control circuit. A controller 130 includes a memory 100 and a plurality of pulse definition circuits (PDC) 110, 120, 122, 124. Four PDCs 110, 120, 122, 124 are shown, though any suitable number may be provided. PDC 110 is shown in detail; the other PDCs 120, 122, 124 may be similar or identical.

PDC 110 includes steering logic 112, aggregate logic 114, and pulse logic 116, each of which interacts with portions of memory 100. The Pulse Logic 116 is configured for determining characteristics of each phase of the pulses to be delivered by the system, and references blocks in a pulse memory 106 where selected parameters for particular pulse phases are stored. Steering logic 112 is configured for determining electrode utilization for pulses to be delivered by the system, and references blocks in a steering memory 102 where selected parameters for particular steering modes are stored. Aggregate logic 114 is configured to obtain pairings of pulses and electrode utilization, and references aggregate memory 104 where selected parameters and definitions of particular combinations are stored. As a result, an aggregate logic instruction will instruct the pulse logic 116 which portions of the pulse memory 106 to access for defining output pulses, and also instructs the steering logic 112 which portions of the steering memory 102 to use for obtaining electrode utilization instructions. The aggregate logic 114 determines as well the sequence and repetition of output pulses to be used. The PDC 110 will determine from configuration memory 108 which aggregate instructions are to be used by the aggregate logic 114.

In operation, at a particular time, PDC 110 will be enabled and receives a command to execute a portion of the instructions stored in the configuration memory 108, with identification of the relevant addresses of the configuration memory 108 to execute. The command received by the PDC may originate from the scheduler 132, which may be part of the microcontroller and/or a separate part of the stimulation circuitry. For example, scheduler 132 may be populated with programming choices made by the physician and includes a schedule of therapy configurations to be delivered with indications of when those therapy configurations are desired to occur, such as hourly, at set times of day, in response to particular sensed conditions (patient standing, sitting, etc.). The contents of scheduler 132 may be populated by the system controller or microcontroller during a time period when the microcontroller is in an awake/active state, and reference the system clock. Once the scheduler 132 is populated with a therapy schedule, the system controller can enter a low power sleep state until an interrupt or programmed wakeup occurs.

The scheduler and PDCs 110, 120, 122, 124 also interact with an arbitration block 134. The arbitration block 134 determines whether or not arbitration rules and relevant holdoffs are satisfied before providing permission to the PDCs to issue therapy.

The contents of the configuration memory 108 determine which portions of the aggregate memory 104 are to be executed, while also carrying additional information. The aggregate logic retrieves the identified portions of aggregate memory 104 in the order prescribed in the configuration memory 108, and uses the retrieved aggregate instructions to instruct the steering logic 112 and pulse logic 116 to obtain instructions for steering and pulses from identified locations in the steering memory 102 and pulse memory 106. The present invention in several examples illustrates how multiple configurations can be linked together, allowing separate PDCs 110, 120, 122, 124 to be linked and operate in a parallel to one another, as detailed further below.

The PDC 110 issues control signals to the electrode combiner 150, which in turn provides control signals to the DAC circuitry 160 and switch matrix 170. The DAC circuitry 160 includes a plurality of current mirrors, referred to as “branches”, the quantity of which determines the resolution of the output signal. Any number of branches or current mirrors can be used. The DAC circuitry 160 can be organized into a number of individual DACs, shown as DAC1, DAC2, etc. For example, two to four, or more, DACs may be provided. As an illustrative example, each DAC circuit could be configured with 100 branches, each providing 1% of the total output current, thereby providing 1% resolution relative to the maximum current of each DAC; the “reference” current can be the current for each branch, and the maximum current of each DAC would thus be the sum of currents for each positive (or each negative) branch in each DAC. The electrode combiner 150 determines how the branches of the DAC circuitry 160 will be combined together for each active electrode of the device, and then instructs the switch matrix 170 which switches are to be opened or closed for allowing the combined branches to be output to the electrodes of the device and/or to which electrodes 180 will be grounded or open-circuited during stimulus output.

Further details as to the hardware that may be used for the various blocks in FIG. 4 can be found in U.S. Pat. No. 10,716,932, the disclosure of which is incorporated by reference. For example, each PDC may be a separate application specific integrated circuit (ASIC), or each may be or include a separate microcontroller or simpler controller circuit. Various logic and switch arrays can be used, as noted above, including switch arrays at the switch matrix 170, and a plurality of current mirrors can be included in the DAC circuitry 160, where ones or sets of the current mirrors may be separately associated with power circuitry to provide headroom for desired current output.

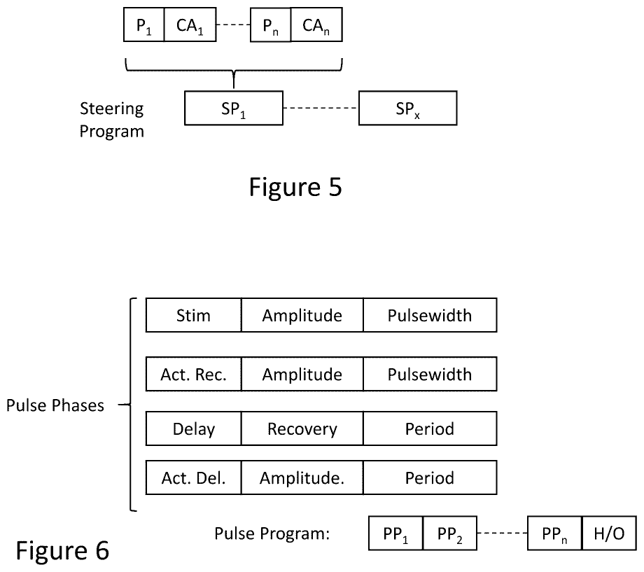

FIG. 5 illustrates a steering program. The memory may hold any desired number of steering programs, such as 8, 16, 32, 64, etc., which are shown as steering programs SP₁ to SPₓ. Each steering program, as illustrated, has memory blocks (at least) for each of the stimulation delivery electrodes of the system. In the illustration, the memory block for the n^{th} electrode includes a polarity definition, Pₙ, and a current allocation definition CAₙ. The polarity definition determines whether the stimulation delivery electrode is an anode or cathode, and may be a single bit in the memory if desired. Polarity definition may use two bits instead, if polarity is defined as any of anode, cathode, ground, or high impedance (which may mean the electrode is available for sensing or is open circuited). Additional bits may be used as desired. The current allocation may use any suitable amount of memory; if resolution of 1% or more is desired, 7 or 8 bits may be used for current allocation for each electrode; fewer bits may achieve lower resolution. Some examples may use a selectable resolution and the steering program may store current allocation for the highest such resolution, with the system simply ignoring one or more bits to provide lower resolution. However the specifics are determined, the illustration shows how each steering program may be stored in memory.

FIG. 6 illustrates a plurality of pulse phase types and an illustrative pulse program. The pulse phases in this illustration have four types: stimulation, active recovery, delay, and active delay. Stimulation phase means that current is being sourced and sunk by the circuitry, using an amplitude defined by the pulse phase and having a duration or pulse width as defined by the pulse phase, and using polarity as defined by a steering program then in use (noting that the aggregate logic defines pairings of pulse program and steering). Active recovery phase also means that current is being sourced and sunk by the circuitry for a duration/pulsewidth, but in this case with a polarity for each electrode being used that is opposite of that defined by a steering program then in use. Delay phase means that no current is actively sourced or sunk by the circuitry for a delay period, and recovery bits are provided that determine whether or not the electrodes of the system are open circuited or grounded, where grounding the electrodes allows passive recovery. During a delay phase the stimulation circuitry can be powered down; an active delay phase is also defined during which no current is actively sourced or sunk, but the stimulation circuitry remains powered on using a specified amplitude during the period defined by the active delay. Active delay phases may be used to prepare for a subsequent stimulation phase.

A pulse program, as shown in the lower part of FIG. 6, defines a sequence of pulse phases PP1, PP₂ . . . . PPₙ. Any number of pulse phases can be included in a single pulse program, and any number of pulse programs may be stored in the pulse program memory. If desired, holdoff instructions may be provided for pulse phases and/or pulse programs, to further the use of arbitration and holdoff logic.

In some examples, once a pulse program starts, each phase of the pulse program is executed sequentially, without any interruption to allow another pulse program or a pulse phase from another program to be executed. This means that a pulse program, once begun, does not stop even if another pulse program or pulse phase of another program is initiated on a different PDC. Alternatively, or additionally, a holdoff setting may be associated with each individual pulse program as a whole, (H/O), allowing the pulse program to be paused in the middle if another pulse program begins executing and calls for a hold-off.

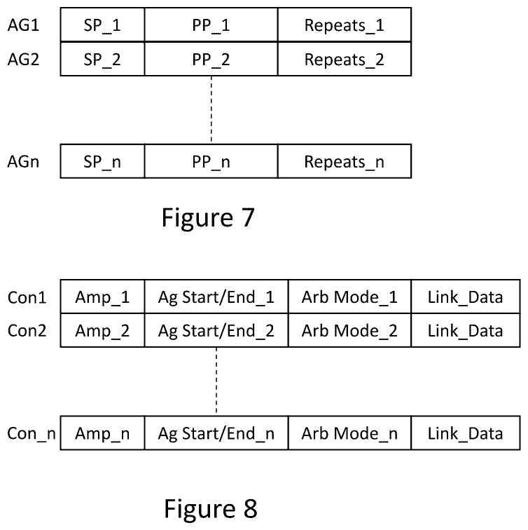

FIG. 7 shows illustrative aggregate instructions. Each aggregate instruction, shown as AG1, AG2 . . . . AGN, identifies a steering program (SP_1, SP_2 . . . . SP_n), and a pulse program (PP_1, PP_2 . . . . PP_n), along with a quantity of repeats of the pulse program to be performed in the aggregate instruction. Holdoff settings may also be included in the aggregate instructions, if desired.

FIG. 8 shows illustrative therapy configurations with arbitration and link data. Each therapy configuration defines an overall amplitude setting (Amp_1, Amp_2 . . . . Amp_n), and defines aggregate instructions to be executed in the therapy configuration. In the example shown, start and end aggregate instructions are identified; in other examples, the aggregate instructions may be identified individually, depending on how the aggregate instruction memory block is organized. In some examples, an arbitration mode can be set at the therapy configuration level, and linking data can be provided for each of the configurations.

Link_data, as used in FIG. 8, may be used to allow more than one therapy configuration to be executed in parallel using two (or more) PDCs (FIG. 4). A linking instruction of this sort can be operated without regard for other arbitration settings, allowing two PDCs to issue output pulses on the same electrodes at the same time. Each PDC may, in this example, access different portions of the DAC circuitry (FIG. 4). For example, the DAC circuitry of FIG. 4 can be defined with two or more separate “DACs” that can be controlled individually by a PDC. Each DAC, in this example, would offer a resolution in the range of, for example, and without limitation, 1% to 10% of total current for the DAC. For example, a given DAC may have 100 branches, which can be grouped together via switch matrices into a number of outputs, where each branch would provide 1% of the total current available from the given DAC. The total current for the given DAC can itself be a controlled parameter, so that each branch would provide 1% resolution relative to the total current for the given DAC.

In some examples, a therapy configuration may identify itself as having instructions for linking two or more PDCs together, if desired. For this reason, FIG. 8 shows link data for each therapy configuration. Alternatively, the overall system scheduler 132 (FIG. 4) may be configured to identify two or more therapy configurations to be operated on simultaneously. When linking information is contained in the scheduler 132, the scheduler 132 may issue commands to first and second PDCs to simultaneously activate first and second therapy configurations. If such a command issues, the link data in the therapy configurations shown in FIG. 8 may be omitted. Each PDC can then be configured to recognize that it has been sent a linked therapy command, which can also be conveyed to the arbitration block 134. In other examples, the microcontroller instead identify two or more therapy configurations to be operated on simultaneously, and so the linking data would be provided to each PDC directly by the microcontroller.

The arbitration block 134 provides a link between PDCs and provides permissions for issuing therapy, such as an enable signal, when all rules regarding holdoff and arbitration are met. When a linked therapy command is generated by the scheduler, the arbitration block 134 uses the linked therapy command to indicate to the two (or more) PDCs that are called by the linked therapy command that permission is given to provide the linked therapy, while still enforcing holdoff and other arbitration commands relative to any other PDC that is not part of the linked therapy command.

Supposing then that a current output of 1.95 mA is desired for a given electrode of a given DAC, the desired current could be provided by using 100% of the branches for the DAC on a single output based on a reference current of 1.95 mA. However, the system is intended to offer much more control than that. For example, some neuromodulation systems allow a clinician programmer to be operated using steering of a central point of stimulation. In such systems, current allocation can be determined with a great deal of specificity, yielding very complex combinations of electrode fractionalization. For example, four electrodes may be used to output current, with the current on each electrode being different from each of the other electrodes, as follows:
Electrode 1: 790 uA (23%)
Electrode 2: 1700 uA (49%)
Electrode 3: 860 uA (25%)
Electrode 4: 110 uA (3%)
   The total current here would be 3.46 mA (milliamps), noting that uA here indicates microamps of current. At 4% resolution, however, the above solution cannot be had and can only be approximated, as follows:
Electrode 1: 830 uA (24%)
Electrode 2: 1660 uA (48%)
Electrode 3: 860 uA (24%)
Electrode 4: 138 uA (4%)
   It may be desirable to allow two DACs each operating at 4% resolution, but with different reference current levels to provide the output current. One solution may be as follows:
Using a first DAC, with reference current of 3.2 mA:
Electrode 1: 768 uA (24%)
Electrode 2: 1664 uA (52%)
Electrode 3: 768 uA (24%)
Electrode 4: 0
Using a second DAC, with reference current of 260 uA:
Electrode 1: 21 uA (8%)
Electrode 2: 31 uA (12%)
Electrode 3: 94 uA (36%)
Electrode 4: 114 uA (44%)
   The final solution then would be as follows:
Electrode 1: Desired 790 uA; Delivered 768 uA+21 uA=789 uA
Electrode 2: Desired 1700 uA; Delivered 1664 uA+31 uA=1695 uA
Electrode 3: Desired 860 uA; Delivered 768 uA+94 uA=862 uA
Electrode 4: Desired 110 uA; Delivered 0 uA+114 uA=114 uA
   The above numerical example is merely illustrative of one way that a solution could be reached. To optimize the current allocation among the two DACs, one approach may be to perform a cost minimization function on a plurality of possible fractionalization solutions, as follows:

$$min {\sum }_{∀ E} \vert ( {i}_{desired} - {i}_{delivered} ) \vert$$

The clinician programmer may be used to determine the current allocations used in the steering programs, and so this cost minimization may be performed as part of the configuration of steering programs. Doing so would take advantage of the higher computing power offered in a clinician programmer, which may be implemented as a tablet or laptop computer. It may be observed that the resulting steering program for each of the two (or more) therapy configurations may end up being quite different if the cost function analysis leads to such a result. Indeed, one of the steering programs may use a given electrode as an anode, and the other as a cathode, if desired, to allow a coarser program to overshoot desired current, and a finer program to correct the overshoot. Thus, not merely fine tuning is offered, but a way to reduce the effects of digital step size on the approximation of a desired output. Other uses of the present concepts may instead use the same or identical steering programs for each of two therapy configurations.

Given the desire to provide improved resolution using an architecture as described, the solution proposed herein is to use the above architecture, with pulse programs, aggregate instructions, and therapy configurations, with the ability to link together more than one therapy configuration. Thus, link data as shown in FIG. 8 may be used to link two therapy configurations together so that both may run simultaneously on separate DAC circuits, while outputting current to the same electrodes. The link data may include for example, an indication that a first therapy configuration is linked to a second therapy configuration, as well as identifying the second therapy configuration. More than two therapy configurations could be linked, if desired, such as by cascading the linking of therapy configurations, where the first therapy configuration identifies the second therapy configuration, and the second therapy configuration identifies a third therapy configuration, and so on. Alternatively, an overall scheduler (see item 132 in FIG. 4) may be used to call two therapy configurations to operate at once as linked therapy configurations, while providing instructions to a system arbitrator (item 134 in FIG. 4) for implementing two therapy configurations simultaneously.

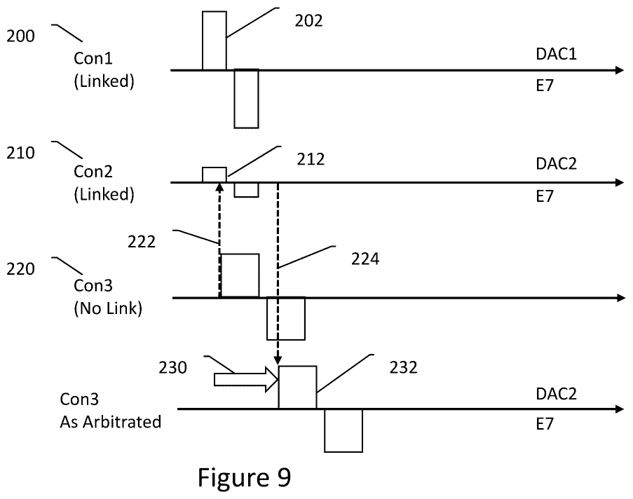

FIG. 9 shows an illustrative example. Here, a first therapy configuration, Con1, is linked to a second therapy configuration, Con2, however, a third therapy configuration, Con3, is not linked to either of the first or second therapy configurations. Therapy configurations Con1 and Con2 may have a holdoff bit set to prevent other therapy configurations from interrupting or being run simultaneously, if desired. Con3, in the example, has an arbitration bit set so that Con3 is required to wait if another therapy configuration is currently operating with a holdoff in force. Each of the horizontal lines in FIG. 9 shows how a given electrode, named E7 in the example, is used in the system.

Con1 and Con2, being linked, are allowed to operate simultaneously and issue pulses 202 and 212 at the same time to the same electrode, E7. Con3, which is not linked, is subject to the holdoff enforced by the linked first and second configurations, and must wait as shown at 230 from the time 222 at which Con3 requests to deliver therapy until the holdoff is released at 224. Then Con3 is allowed to use electrode E7 to issue the stimulus.

Anther way of understanding FIG. 9 would be if Con2 was using a particular one of several available DACs, indicated as DAC2 in the Figure. The holdoff enforced by the linked therapy configurations Con1 and Con2 means that DAC2 is not available at time 222 to generate a therapy pulse as requested by Con3. As a result, Con3 must wait, as shown, even if Con3 was not requesting to use the same electrode, E7, as the other therapy configurations. Pulses 202 and 212 are issued at the same time and on the same electrode, while pulse 232 is pushed back in time as shown at 230 before executing.

The solution shown in FIG. 9 allows the two therapy configurations, Con1 and Con2, to operate at the same time, using two different DACs, DAC1 and DAC2, to issue current through the same electrodes, such as E7 as illustrated. By setting each DAC to a different current level, one DAC can be used to provide a gross approximation, and the other a finer approximation, of the desired current level. Meanwhile, the linked configurations can still enforce arbitration and/or holdoffs relative to other therapy configurations. It may be noted that “arbitration” refers to whether a requested pulse delivery, such as a pulse of a therapy configuration, needs to request permission before being issued, while holdoff refers to whether an ongoing pulse delivery prevents other therapy configurations, aggregate instructions, or pulse programs, from being executed using the same DAC or the same electrodes.

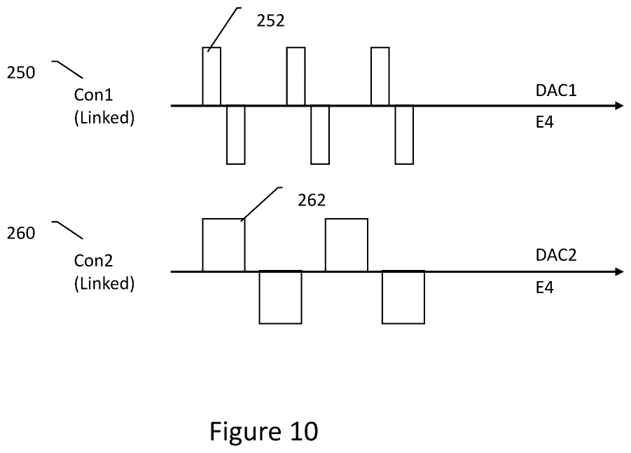

FIG. 10 shows another illustration of how two therapy configurations can be linked together. Here a first therapy configuration, Con1, is linked to a second therapy configuration, Con2. The link between the two therapy configurations may be a stored as part of either therapy configuration Con1 or Con2. Alternatively, the scheduler for the system may be configured to identify Con1 and Con2 as linked together, and calls both to operate at the same time, while providing instructions or other markers to the system arbitrator identifying the two configurations as linked.

As a result, both operate simultaneously, using separate DACs, DAC1 and DAC2. Both therapy configurations can issue pulses from their respective DACs to any of the electrodes available in the system. If, as indicated, both are delivered to one electrode, E4, a rather complex pattern for stimulation would result from a fairly simple definition of the two pulse programs and therapy configurations. This additional flexibility may be advantageous to simplify programming tasks.

One note with FIG. 10 is that the therapy holdoff may be defined by the therapy configuration that takes longest to complete execution. In the example, Con1 issues a repeating pulse program (for example, by calling an aggregate instruction having two repeats), and Con2 issues a different repeating pulse program (for example, calling an aggregate instruction which uses a pulse program having two stimulation pulses, two active recovery pulses, and several delay or active delay periods). Holdoff can be enforced by the therapy configuration that takes the longest to complete. This rule may be handled by ensuring that any linking instruction is provided on the longest-duration of two linked therapy configurations, for example.

Each of these non-limiting examples can stand on its own, or can be combined in various permutations or combinations with one or more of the other examples. The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. An implantable medical device comprising:
a housing (10) containing a power source (32), a controller (30, 130), and stimulation circuitry (34, 150), the stimulation circuitry including at least first and second digital-to-analog converters (DACs); and
a lead (20) having a plurality of electrodes (22) thereon, the lead coupled to the housing such that the stimulation circuitry can issue stimulus pulse patterns to a patient via the electrodes; wherein the controller comprises:
a memory (100) including steering memory (102), aggregate memory (104), pulse memory (106), and configuration memory (108);
a plurality of pulse definition circuits (110, 120, 122, 124) each including steering logic (112), aggregate logic (114), and pulse logic (116);
wherein the steering memory contains steering instruction sets for a plurality of steering programs, each steering program determining which of the electrodes receive a fraction of a total stimulus output current, and the steering logic is configured to implement a selected steering instruction set;
wherein the pulse memory contains pulse programs, each having a one or more pulse instructions defining pulse components each having a pulse type and one or more determining characteristics for the pulse type;
wherein the aggregate memory contains aggregate instructions each defining one or more aggregated outputs, each aggregated output pairing a selected steering instruction set with a selected pulse program and defining a number of repetitions for the selected pulse program to execute with the selected steering instruction set;
wherein the configuration memory defines a plurality of therapy configurations each having a defined total stimulus output current, an arbitration mode, a holdoff setting, and linking data, and identifying a one or more aggregate instructions to be executed for each therapy configuration;
further wherein the linking data in the configuration memory enables two therapy configurations to operate simultaneously, using separate DACs and any of the electrodes.

2. The implantable medical device of claim 1, wherein the linking data of a first therapy configuration identifies a second therapy configuration with which the first therapy configuration is linked.

3. The implantable medical device of claim 2, wherein the first therapy configuration calls for issuance of a first therapy pulse from a first DAC having a first amplitude via a first electrode, and the second therapy configuration calls for issuance of a second therapy pulse simultaneous with the first therapy pulse, from a second DAC having a second amplitude via the first electrode.

4. The implantable medical device of claim 3, wherein the first therapy configuration requests a first total current from the first DAC, and the second therapy configuration requests a second total current from the second DAC, wherein the first total current is larger than the second total current.

5. The implantable medical device of claim 2, wherein the first therapy configuration calls for a first pulse train having first parameters, and the second therapy configuration calls for a second pulse train having second parameters, the first parameters being different from the second parameters.

6. The implantable medical device of claim 1, wherein the stimulation circuitry comprises a plurality of digital-to-analog converter circuits including selectable current mirrors, and the total output amplitude is defined in terms of total output current, such that the implantable medical device is configured to deliver current controlled neural stimulation.

7. The implantable medical device of claim 6, wherein the pulse definition circuit is coupled to the plurality of digital-to-analog converter circuits and is configured to instruct a selected one of the plurality of digital-to-analog converter circuits to use the total output current, and divide the total output current using the selected steering instruction set.

8. The implantable medical device of claim 6, wherein the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

9. An implantable deep brain stimulation system comprising the implantable medical device of claim 1, and a clinician programmer adapted to communicate with the implantable medical device and program each of the arbitration mode and holdoff settings stored in the implantable medical device; wherein the lead is adapted for placement in the brain of a patient.

10. A spinal cord stimulation system comprising the implantable medical device of claim 1, and a clinician programmer adapted to communicate with the implantable medical device and program each of the arbitration mode and holdoff settings stored in the implantable medical device; wherein the lead is adapted for placement in the spinal column of a patient.

11. An implantable medical device comprising:
a housing (10) containing a power source (32), a controller (30, 130), and stimulation circuitry (34, 150), the stimulation circuitry including at least first and second digital-to-analog converters (DACs); and
a lead (20) having a plurality of electrodes (22) thereon, the lead coupled to the housing such that the stimulation circuitry can issue stimulus pulse patterns to a patient via the electrodes; wherein the controller comprises:
a memory (100) including steering memory (102), aggregate memory (104), pulse memory (106), and configuration memory (108); and
a plurality of pulse definition circuits (110, 120, 122, 124) each including steering logic (112), aggregate logic (114), and pulse logic (116);
wherein the steering memory contains steering instruction sets for a plurality of steering programs, each steering program determining which of the electrodes receive a fraction of a total stimulus output current, and the steering logic is configured to implement a selected steering instruction set;
wherein the pulse memory contains pulse programs, each having a one or more pulse instructions defining pulse components each having a pulse type and one or more determining characteristics for the pulse type;
wherein the aggregate memory contains aggregate instructions each defining one or more aggregated outputs, each aggregated output pairing a selected steering instruction set with a selected pulse program and defining a number of repetitions for the selected pulse program to execute with the selected steering instruction set;
wherein the configuration memory defines a plurality of therapy configurations each having a defined total stimulus output current, an arbitration mode, a holdoff setting, and identifying a one or more aggregate instructions to be executed for each therapy configuration;
the implantable medical device further comprising a scheduler identifying therapy configurations to be executed and times at which the therapy configurations are to be executed, wherein the scheduler is configured to identify a first therapy configuration as linked to a second therapy configuration, and to instruct the pulse definition circuits to simultaneously execute the first therapy configuration on a first DAC, and the second therapy configuration on a second DAC.

12. The implantable medical device of claim 11, wherein the first therapy configuration calls for issuance of a first therapy pulse from the first DAC having a first amplitude via a first electrode, and the second therapy configuration calls for issuance of a second therapy pulse simultaneous with the first therapy pulse, from the second DAC having a second amplitude via the first electrode.

13. The implantable medical device of claim 12, wherein the first therapy configuration requests a first total current from the first DAC, and the second therapy configuration requests a second total current from the second DAC, wherein the first total current is larger than the second total current.

14. The implantable medical device of claim 11, wherein the first therapy configuration calls for a first pulse train having first parameters, and the second therapy configuration calls for a second pulse train having second parameters, the first parameters being different from the second parameters.

15. The implantable medical device of claim 11, wherein the stimulation circuitry comprises a plurality of digital-to-analog converter circuits including selectable current mirrors, and the total output amplitude is defined in terms of total output current, such that the implantable medical device is configured to deliver current controlled neural stimulation.

16. The implantable medical device of claim 15, wherein the pulse definition circuit is coupled to the plurality of digital-to-analog converter circuits and is configured to instruct a selected one of the plurality of digital-to-analog converter circuits to use the total output current, and divide the total output current using the selected steering instruction set.

17. The implantable medical device of claim 15, wherein the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

18. An implantable deep brain stimulation system comprising the implantable medical device of claim 11, and a clinician programmer adapted to communicate with the implantable medical device and program each of the arbitration mode and holdoff settings stored in the implantable medical device; wherein the lead is adapted for placement in the brain of a patient.

19. A spinal cord stimulation system comprising the implantable medical device of claim 11, and a clinician programmer adapted to communicate with the implantable medical device and program each of the arbitration mode and holdoff settings stored in the implantable medical device; wherein the lead is adapted for placement in the spinal column of a patient.

20. A method of operation in an implantable neurostimulation device, the method comprising:
calling for execution of a first therapy configuration according to a schedule, the first therapy configuration identifying a second therapy configuration as being linked thereto;
executing the first therapy configuration on a first digital-to-analog converter (DAC) of the implantable neurostimulation device; and
simultaneous to executing the first therapy configuration on the first DAC, executing a second therapy configuration on a second DAC; wherein:
the first DAC, operating in accordance with the first therapy configuration, issues a first pulse using a first electrode of the implantable neurostimulation device during a first time period; and
the second DAC, operating in accordance with the second therapy configuration, issues a second pulse using the first electrode during the first time period, such that the first and second pulses combine together as a single output pulse.
